(19) United States
(12) Patent Application Publication
(10) Pub. No.: US 2006/0281913 A1
(43) Pub. Date: Dec. 14, 2006

(54) Process for the production of crystalline xylose from sugar cane bagasse, crystalline xylose obtained by said process, process for the production of xylitol from the said xylose and crystalline xylitol obtained thereby

(76) Inventors: Joao Afonso Ferreira, Niteroi (BR); Claudio Octavio Teixeira, Rio De Jeneiro (BR); Sergio Murilo Soares, Rio De Jeneiro (BR)

Correspondence Address: BIRCH STEWART KOLASCH & BIRCH, PO BOX 747, FALLS CHURCH, VA, 22040-0747, US

(21) Appl. No.: 10/556,589

(22) PCT Filed: May 26, 2004
(86) PCT No.: PCT/BR04/00078

§ 371 Date: Nov. 14, 2005

(30) Foreign Application Priority Data
Jun. 10, 2003 (BR) PI0301678-1

(51) Int. Cl. C07H 1/02

(52) U.S. Cl. 536/124

(57) ABSTRACT

The invention relates to a process for the production of crystalline xylose, from sugar cane bagasse, which xylose consists of microcrystals having a well-defined morphology and a narrow granulometric range, a further object of the invention being the protection of the product, crystalline xylose, manufactured according to the technological route which is detailed in the text of the patent. Additionally, the invention also relates to a process for obtaining crystalline xylitol with particularly adequate physical and functional characteristics, from the xylose produced according to the process idea conceived by the Applicant, as well as to the product proper, crystalline xylitol, generated in accordance with a particular sequence of unit operations. The process in question comprises an initial step for the formation of the xylose by means of an acid hydrolysis of the bagasse; a second step in which there is achieved the purification of the xylose solution thus formed; a third step in which there is accomplished the crystallization of the xylose by a controlled cooling of this said aqueous solution, thereby producing crystals with a xylose content higher than 99.0% by weight, on a dry solids basis; a fourth step which consists in the hydrogenation of the xylose and its consequent conversion to xylitol; a fifth step for the treatment and evaporation of the xylitol solution; and a last step for the crystallization of the xylitol, which is also carried out by a controlled cooling, thereby leading to the manufacture of crystals with a xylitol content which is never lower than 99.5% by weight, on a dry solids basis. The crystalline xylose and the crystalline xylitol produced according to the invention have remarkable degrees of purity, as pointed out, and exhibit, besides an excellent fluidity, a hygroscopicity and a dissolution time which confer on them an optimum performance when utilized in their industrial applications.

The present invention relates to a process for the production of crystalline xylose of a high chemical purity through crystallization thereof, which is accomplished by the controlled cooling of an aqueous solution of xylose, said xylose being obtained from sugar cane bagasse. The said xylose, prepared by the process according to the invention, is composed of well-defined crystals, and is constituted of particles which exhibit a narrow size distribution with a mean diameter comprised between about 150 microns and about 300 microns. The physical and functional characteristics of the crystalline xylose according to the present invention, such as purity, granulometric size distribution and mean diameter of the crystals, apparent density, hygroscopicity, and dissolution time, are adjusted in order to maximize its performance in specific industrial applications. Accordingly, another object of the invention is to provide the protection of the product, crystalline xylose, manufactured in accordance with the process of the invention. Still another object is to provide the protection of the crystalline xylitol, obtained from this said xylose, as well as of the corresponding preparation routes.

The technical field of the invention is concerned with naturally occurring nutritive sweeteners, the most important of these being the sucrose, the glucose, the fructose and the xylose proper, which are saccharides produced on a large industrial scale and widely consumed as simple sugars or as ingredients in several edible products.

Xylose is a pentose possessing the molecular formula C₅H₁₀O₅, which can be obtained from hemicellulosic vegetable materials containing xylans, having a low calorific value and exhibiting a sweetening power, according to some data collected from the literature, approximately equivalent to 67% that of sucrose. These features render the xylose particularly suitable to be employed as sweetener, additive excipient or preservative in several food and beverage products, especially the dietetic ones and those directed to diabetics. The aforementioned features, coupled with their good influence on the treatments for coronary and digestive system diseases, and for hypertension, open up the possibility that xylose may be used as raw material in the formulation of various medicines.

Another property of the xylose which contributes to the success of its industrial applications relates to its proven ability to prevent the appearance of tooth decay. However, despite the aforesaid potential uses, it is as a raw material for producing xylitol that xylose has been generally recognized in the international market.

Crystalline xylose is composed of white crystals which are odorless and slightly hygroscopic, with a density of 1525 kg/m³ and a melting point of between 152 and 154° C., and are sparingly soluble in methanol, ethanol and isopropanol, and highly soluble in water. It should also be noted that, according to the technical publications Food Chemicals Codex (1992) and The United States Pharmacopeia (1990), xylose is defined as containing not less than 98% by weight on a dry solids basis of the substance xylose (C₅H₁₀O₅).

As regards the crystalline xylitol, it is a polyol possessing the molecular formula C₅H₁₂O₅, which is found in very small quantities in plants, fruits and vegetables, and which aspect hinders its economical utilization from such sources, which also stands out owing to its reduced calorific content and a sweetening power equivalent to that of sucrose, thereby rendering it suitable to be used as sweetener in the pharmaceutical and the food and beverage industries.

Another special particularity of the xylitol concerns its effective action to prevent—and, according to the most recent researches, even to avoid—the appearance of dental caries, thereby leading to the recommendation for its use as the ideal substitute for the sucrose in human nutrition, and as a particularly important substance for the preparation of toothpaste. Moreover, due to its easy and insulin-independent metabolism in the body, xylitol emerges as a highly appropriate ingredient to be used in foods for diabetics, without harming the calorific requirements recommended by the experts.

Crystalline xylitol is constituted of crystals which are white, odorless and hygroscopic, having a negative heat of solution of about −34.8 cal/g, due to which there is felt the pronounced freshness when it comes into contact with the saliva, having a density of 1520 kg/m³ and a melting point of between 92 and 96° C., and exhibiting a low solubility in methanol, ethanol and isopropanol, and a high solubility in water. It should also be noted that, according to the technical publications Food Chemicals Codex (1992) and The United States Pharmacopeia (1990), xylitol is defined as containing not less than 98.5% by weight on a dry solids basis of the substance xylitol (C₅H₁₂O₅).

In large part, the processes for the manufacture of xylose which have been patented since the 1970s adopt chemical routes, particularly those based on the hydrolysis of biomass containing an appreciable amount of xylans. Thus it is that the U.S. Pat. No. 3,990,904, the U.S. Pat. No. 4,075,406 and the U.S. Pat. No. 4,226,638 employ, as raw material, residues from the pulp and paper industry, wood chips, corn cobs and other kinds of vegetable, in order to obtain, through a preliminary step of acid hydrolysis, a solid waste containing lignin and a xylose solution which is capable of being purified by means of a subsequent treatment.

In line with the technological improvement of the aforesaid route, aiming at the processing of hemicellulosic material with considerable amount of xylans, some recent patents combine classical separation unit operations in an attempt, complex and not always well succeeded, to improve the yield of xylose recovery from the associated solid waste (U.S. Pat. No. 5,340,403 and U.S. Pat. No. 6,086,681), while other ones invest in chromatographic separation processes with the purpose of obtaining a xylose solution relatively pure and capable of, by means of a subsequent controlled crystallization, yielding crystalline xylose within the range of the desired specifications (U.S. Pat. No. 5,084,104 and U.S. Pat. No. 6,239,274).

However, in the 1980s and the 1990s several research groups directed their work, in the field of the production of xylose, towards the biochemical routes, employing microorganisms to the manufacture of enzymes adequate to effect the hydrolysis of hemicellulosic substrates containing xylo-oligomers as shown in the approaches adopted by U.S. Pat. No. 4,200,692, U.S. Pat. No. 4,275,159 and U.S. Pat. No. 5,932,452. Such effort, according to their main researchers, is justified due to the fact that it leads to, without the need for intermediate and exhaustive steps of purification of the xylose hydrolysate, the attainment of a final product with purity and yield values equivalent to those attained through the classical chemical routes.

This same division of xylose production between chemical and microbiological alternatives was preserved with respect to the ensuing manufacture of xylitol, as it is shown in a survey of the prior art carried out on this subject. In the first group of processes, returning analogously to the 1970s, various patents reveal strategies for the synthesis of xylitol based on, in an initial step of the processing, the acid hydrolysis of wood or other kinds of vegetable material, in order that the xylose thus obtained, after the conclusion of the required purification steps of the hydrolysate generated, be directly hydrogenated, leading to the formation of the intended crystalline xylitol, following the completion of some complementary operations typical of each one of the manufacturing processes described in these documents (U.S. Pat. No. 3,970,712 and U.S. Pat. No. 3,980,719).

Shortly afterward, assuming that the reaction of catalytic reduction of the xylose to xylitol had been mastered, several other studies concerning this same line of thought began to focus on particular aspects of the manufacturing process. For instance, with the main purpose of increasing the final purity of xylitol and adjusting it to the stringent consumer's requirements, the U.S. Pat. No. 3,985,815 delves into the exploration for the ideal conditions for fractional crystallization of xylitol solutions resulting from the hydrogenation of the xylose. The U.S. Pat. No. 4,008,285, which is more comprehensive, explores all the lengthy conversion of hemicellulosic raw materials into crystalline xylitol, without neglecting the purification of the acid xylose solution resulting from the hydrolysis, but dwelling on the analysis of the utilization of ion-exchange resins to enrich, through chromatographic separation techniques, both this xylose hydrolysate and the xylitol solution resulting from the xylose hydrogenation.

Other interesting development works which search for some specific answers related to inconveniences arising from the use of distinct industrial wastes, characterized by their significant content of xylo-oligomers, but with high levels of contaminants which are undesirable during the processing of xylitol, illustrate furthermore the appearance of technological innovations in the routes which are purely chemical in character, as it is the case of the U.S. Pat. No. 5,998,607, which introduces a method for producing xylitol from a xylose solution having a high and undesirable content of xylonic acid.

Adopting the biochemical route for the production of xylitol, analogously to the aforementioned case of the xylose, some researchers concentrated their efforts, in the last two decades, on developing microorganisms capable of effecting the biosynthesis of xylitol, using as the starting material a solution of xylose. The variations in such approach can be appreciated in some American patents (U.S. Pat. No. 5,081,026, U.S. Pat. No. 5,631,150 and U.S. Pat. No. 6,271,007) as well as in Brazilian ones (Pat. BR 9,007,009 and Pat. BR 9,105,939), the majority of which highlighting an additional advantage, besides dispensing with consecutive product purification stages, said advantage being the production of ethanol as a by-product at the end of the reaction, owing to the transformation of a large part of several other hexoses which are present in the medium. Other remarkable publications on this issue concern the microbial synthesis of xylitol from glucose, by means of the aerobic fermentation of this said glucose to arabitol, followed by its oxidation to xylulose and the enzymatic isomerization of this said xylulose to xylose, in order that only then be carried out the catalytic hydrogenation for the conversion of the xylose to xylitol (Pat. BR 9,004,978 and U.S. Pat. No. 6,221,634).

In view of the assessed picture, it becomes evident that the processes for the production of crystalline xylose that have already been patented—and, consequently, the predominance of those which yield the crystalline xylitol—combine several processing steps having considerable complexities, in both the chemical route and the biochemical route, which bring about an array of technical difficulties arising from the unsatisfactory efficiency and productivity of the process, thereby contributing to the manufacture of a final product, be it crystalline xylose or crystalline xylitol, with occasionally high production costs.

As regards the chemical route, an appraisal of the literature of the prior art shows that there are undeniable obstacles to attaining a significant yield in the extraction of a xylose solution with a desirable purity, using any hemicellulosic raw material as a starting point, mainly due to the losses caused by the set of purification treatments required for the elimination of contaminants, since so many combined separation unit operations, carried out in accordance with the standard procedures established in the reference books, finally result in hindering definitely the process economics.

As for the real expectations of the biochemical route, equally unquestionable are the major inconveniences connected with the utilization of microorganisms which are pronouncedly sensitive to the process and operating conditions required in the industrial plants, as it is so well revealed by the patents adopting this alternative processing approach, and which compose the prior art.

Moreover, besides the expressive set of barriers, often not surmountable, brought on by technical particularities concerning selectivity and yield in the practice of adopting the microbial way, it is undeniable that imperative purification needs arise as definitive elements which contribute to the aggregation of additional high production costs, thereby eliminating competitive advantages, which are nearly always preliminarily and hastily stated as belonging to this said strategy for obtaining the final product, being it either crystalline xylose or crystalline xylitol, when compared to the chemical route.

Therefore, in view of the above considerations, it is inferred that there exists a concrete lack of a processing route for the production of crystalline xylose and crystalline xylitol which, at the same time, can embody all the essential positive aspects of interest, according to the comments recorded in the several different patents that have already been published. The gap to be filled refers to a technology capable of minimizing the losses resulting from subsequent purification steps, since there will always remain a waste of biomass to be separated from the main stream, without neglecting the expected yield of the overall process and the growing demands of final product purity determined by the market, but it also refers to an urgency for the rationalization of costs in the concatenation of multiple processing stages, starting from the desirable utilization of low-priced and abundant raw material up to the integration of unit processes and operations conveniently optimized to maximize its final transformation.

The present invention has the feature of embodying a large number of advantageous characteristics in the coordinated production of crystalline xylose and crystalline xylitol, when compared with the several alternatives existing in the prior art. In the foreground, with undisputed adequacy, the process which was conceived uses sugar cane bagasse, which is a hemicellulosic material that had not yet been suggested in the documents that have been cited in the prior art, as a raw material, utilizing rationally a waste which is plentiful and inexpensive in the sugar mills and, consequently, becoming integrated into an industrial sector of long tradition in the Brazilian economy.

Furthermore, due to the operating conditions employed in the preliminary phases of the bagasse treatment, the processing route belonging to this invention entails reduced steps of purification of the xylose solution obtained, which exhibit material losses much smaller than those described in the previously mentioned patents of interest, and which are devoid of any increase of negative interference in the computation of the efficiency of the extraction of the xylose existing in the bagasse, these features constituting facts that emerge as contributory elements in order that be possible to attain high manufacturing process, overall yields, determined by the proportion by weight between the final product and the bagasse fed into the system.

Additionally, the subsequent unit operations and processes, involving the crystallization of the xylose and its ensuing transformation to xylitol, ensure excellent degrees of product purity at the end of the processing, thereby rendering the invention especially differentiated for incorporating results which are very positive with regard to the binomial yield and purity. This occurs, it is important to emphasize, without any harm to the manufacture of a crystalline xylose and a crystalline xylitol perfectly adjusted to meet the needs of the multiple industrial sectors in which they are used, not only when their physicochemical requisites are analyzed, but also when the morphological aspects required for their applications are assessed.

Besides having such a set of significant advantages mentioned above, the present invention, in regard to the process for the production of crystalline xylose and crystalline xylitol proper, is also markedly distinguished from the other processes described in the prior art, and possesses a remarkable inventiveness which is made evident by the sequence of unit operations, despite each of these being known in generic terms, and by the adjustment of certain conditions in each one of these unit operations. Such inventiveness, it should be stressed, is revealed through various technical improvements and innovations which combine to form a processing route which is simpler and more efficacious when it is compared with the patented technologies, and which route consists of a first step, of an essentially chemical character, wherein the xylose is formed by means of the acid hydrolysis of bagasse; a second step wherein the xylose solution extracted is purified; a third step wherein the crystallization of xylose is carried out by the controlled cooling of its aqueous solution which has been previously concentrated; a fourth step wherein the xylose is hydrogenated to produce a xylitol solution; a fifth step wherein this said solution is treated and evaporated; and a sixth step wherein, similar to the xylose, the crystals of xylitol are also formed through the controlled cooling of its aqueous solution.

The crystalline xylose produced by the process according to the invention has a high degree of purity, which is quantified by a xylose content higher than 99%, while the crystalline xylitol derived therefrom also has an excellent purity, expressed in terms of the xylitol content, never lower than 99.5% (both percentages by weight on a dry solids basis).

The excellent purity of the xylose and xylitol crystals, their well-defined shapes and the significant uniformity of their sizes result in a very good stability of the final products, which in turn is manifested by a reduced hygroscopicity. Also, in consequence of the said purity level and the morphological integrity of the crystals produced by the process according to the invention, the particulate materials in question possess a low friability which permits that a good flow index be maintained during their handling, packing, storage and even during the ultimate consumption. Moreover, their microcrystalline granules exhibit a narrow particle size distribution with a mean diameter equivalent to 200 microns, in regard to the xylose, and to 450 microns, with regard to the xylitol, thereby bringing about a good fluidity of the powder and contributing to the achievement of short dissolution times of their constituent particles.

In view of the facts discussed above, it is possible to conclude that the manufacture of crystalline xylose and crystalline xylitol, from sugar cane bagasse, having a high chemical purity and with the other properties conveniently adjusted so as to satisfy stringent requirements for application in different industrial sectors, through a route which exhibits costs relatively reduced and which has an overall yield maximized, becomes viable owing to the technical novelties and improvements incorporated into the manufacturing process developed and optimized in this invention.

In summary, the present patent keeps in its broad kernel, as main objects, the following aspects, supported by studies, experiments, and laboratory and industrial tests carried out in the facility of the Applicant: a) the process for the production of crystalline xylose of high purity utilizing sugar cane bagasse as raw material, with the relevant features previously mentioned, which render it entirely original with respect to the prior art; b) the crystalline xylose resulting from the corresponding process; c) the subsequent complementary processing, in which the starting material is the xylose thus generated, to obtain crystalline xylitol of high purity with intrinsic advantages which truly reinforce the innovative character of this patent; d) the crystalline xylitol derived from the composition of the six steps of the technological route briefly discussed previously and which will be better described, with all its chief aspects, in the following paragraphs of this document.

One of the fundamental ideas that guided the development of the preliminary work regarding this invention consisted in establishing an original and commercially viable technological route using sugar cane bagasse as raw material, inasmuch as it is relatively cheap, available on a large scale in the Brazilian territory, and very reliable with regard to its supply, with an adequate standard of quality, so that it can be employed as an industrial feedstock.

However, a problematic aspect inherent in the typical processes for the manufacture of the xylose and the xylitol, described in the prior art, that must be stressed to underline even better the advancements related to the present patent, refers to the difficulty in obtaining a final product, be it either crystalline xylose or crystalline xylitol, with simultaneously satisfactory levels of purity and yield, without the imperative inclusion of critical operations, which are too complex and costly, in the purification of the process streams.

The researches which were undertaken by the Applicant revealed undoubtedly that the strategic utilization of the aforementioned raw material in question, despite the big advantages previously pointed out, enables the minimization of the number of purification steps for the xylose solution extracted from the bagasse, with the consequent reduction of the material losses which are very common in this stage of elimination of contaminants and other undesirable substances for the processing, but without harming the purity requirements determined by the final consumers of xylose and xylitol in their usual applications. The pure nonexistence of chromatographic separation units in the route conceived herein, already represents a tremendous reduction in capital investment in equipment for the assemblage of an industrial plant, as well as a remarkable factor in the reduction of the costs associated with its operation, especially due to the processing of more concentrated streams, which in turn do not require additional consumption of energy for its evaporation.

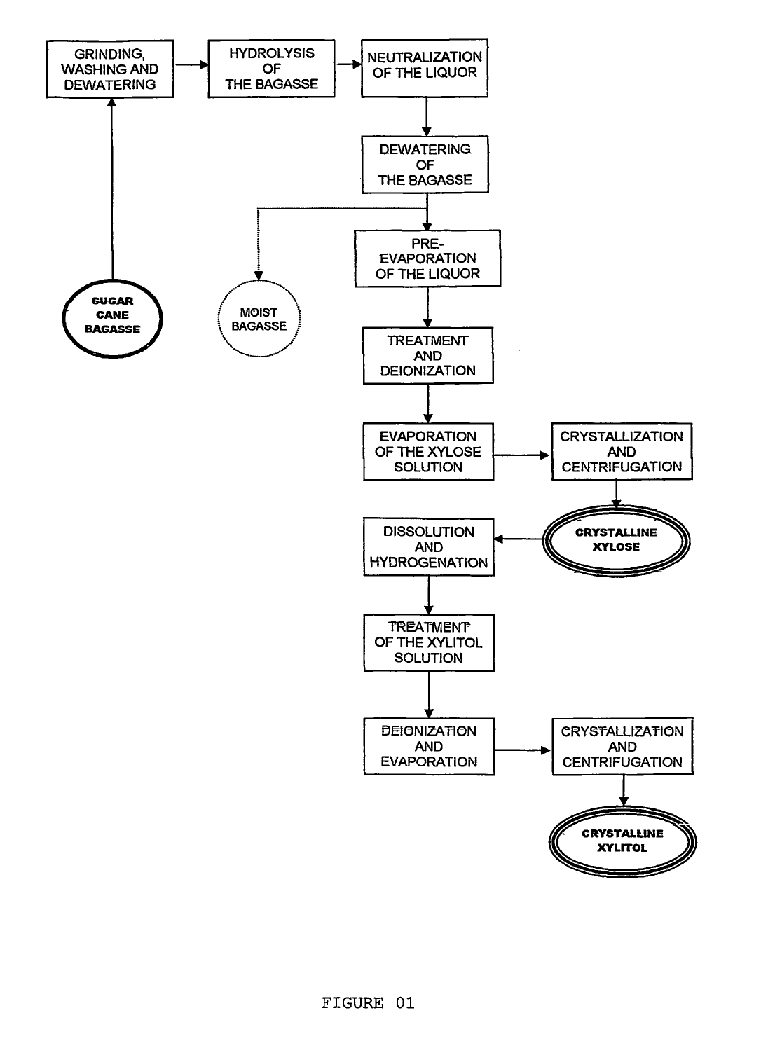

The overall process for the production of crystalline xylose and crystalline xylitol, both of high purity, utilizing sugar cane bagasse, said process comprising an object of the present invention, is discussed below in detail and shown schematically in FIG. 1, and which process comprises six distinct steps, which are individually described for a better understanding of their particularities as follows: the first which ends with the hydrolysis of the vegetable material; the second wherein the xylose extract is purified; the third wherein takes place the crystallization of xylose; the fourth in which, by means of a hydrogenation reaction, the xylose is transformed to xylitol; the fifth which is employed for the treatment and the evaporation of the aqueous solution of xylitol obtained previously; and the sixth wherein the crystals of xylitol are generated through the controlled lowering of the temperature of its aqueous solution.

The first step, after receiving the sugar cane bagasse from the sugar mill, is initiated by grinding this raw material in a rotary knife cutter mill, adjusted to effect a substantial reduction in the size of the fibrous material, so that no more than 5% of the resultant particles exhibit a size greater than 3 millimeters. This measure is vital to increase the superficial area of this solids fraction, and, as a consequence, to favor a thorough contact between the phases in the subsequent moments of the processing, mainly during the acid attack to the hemicellulosic matrix in the course of the hydrolysis.

Thereafter, aiming to remove residual impurities from the finely divided bagasse, already aggregated with the raw material at the moment when it was acquired from the mill, the solid feed is subjected to a washing operation with hot water at a temperature of at least 80° C., with an approximate weight ratio of water to bagasse being comprised between 5 and 10, in tanks with permanent and vigorous agitation, in order that the turbulent effects of the system intensify the action of removal of the debris contained in the organic material, thereby freeing it of sediment, sand and other foreign bodies.

Finally, in this stage related to the preliminary treatment of the bagasse, the suspension is transferred to dewatering presses, where the aqueous fraction is isolated, thereby carrying the residual impurities and freeing the vegetable substrate from interfering substances harmful to the subsequent physicochemical processes.

The formation of the xylose occurs as a result of the acid hydrolysis of the hemicellulosic material with a high content of xylan, present in the sugar cane bagasse, according to carefully adjusted process conditions. The bagasse deriving from the dewatering is fed into a stirred hydrolysis reactor, with a previous amount of water ballast, thus forming a suspension with a solids content, by weight, comprised between 10% and 20%. Thereafter, at a temperature of between 120° C. and 150° C. and for about 120 minutes, the said xylan undergoes a hydrolysis reaction catalyzed by sulfuric acid, which is added to the water, prior to the introduction of the bagasse, as a solution having a concentration of 98%, in such a proportion so as to adjust the pH within the range of 1.0 and 2.0, thereby yielding a xylose solution having a total dry solids concentration of between 2% and 6% by weight, and a xylose purity within the range of 60% and 75% by weight, on a dry solids basis.

The crux of this hydrolysis reaction lies in the rigorous control of the operating conditions, in accordance with the procedures followed in the present invention, in order that, despite being reached a maximum yield for the xylose extraction, do not occur a parallel degradation of the lignin and the cellulose present in the vegetable material, remaining such compounds in the insoluble residue.

It is of interest to note that, in the absence of a meticulous investigation capable of determining the optimum set of the reaction process variables, if the conditions employed to accomplish the hydrolysis should also permit the generalized decomposition of all the ligno-cellulosic content of the bagasse, as well as of other carbohydrates contained therein, the subsequent purification of the xylose solution will require various additional efforts for treatment, as described in many patents constituting the prior art, thereby raising too much the manufacturing costs in order that be attained, when effectively viable, the purity required by the several different applications (U.S. Pat. No. 5,340,403 and U.S. Pat. No. 6,086,681).

Confirming some of the concepts enunciated herein previously, it is important to emphasize once more that, among the innovations introduced by the Applicant into its technological development work, the one which deserves a special mention concerns the establishment of optimized parameters in the step of hydrolysis of the bagasse, thereby reducing, as it can be seen in the following paragraphs, the complexity typical of the purification units included in the so-called chemical routes for the production of xylose and of xylitol.

The second step of the processing, consisting in the purification of the hydrolyzed liquor, is initiated through its neutralization, by means of the use of a suspension of calcium hydroxide with a concentration of between 5% and 10%, to adjust the pH of the medium in the range of 6.0 to 7.0. Thereafter, the liquor is dosed with ferric chloride and an anionic polyelectrolyte, thereby obtaining important combined effects of precipitation and flocculation, which act to increase the efficiency of removal of the impurities of the liquor, thus eliminating its turbidity, as well as excluding a large part of the colored corpuscles perceived in the liquid phase and thereby attenuating drastically the intensity of its dark color.

Then there remains, in another dewatering operation, to promote the separation of the partially purified liquor from the aforesaid solid residue which is made up, fundamentally, of the processed bagasse, but to which is also adhered a significant portion of the contaminants which have been already removed from the liquid phase. With this procedure, there is obtained, as an accessory advantage, a bagasse which is moist and neutralized, capable of being returned to the sugar mill, wherein it can be used directly as fuel in the steam generation units, thereby rationalizing the integrated energy balance of the mill and of the xylose plant, without the risk of feeding substances that are harmful to the internal walls of the boilers.

Afterward, the partially purified liquor, with a dry solids content of between 2% and 6% by weight and a xylose purity within the range of 60% and 75% by weight, on a dry solids basis, passes through an evaporation unit, operating under a vacuum in the range of 700 to 750 mm Hg, wherein its concentration, due to the loss of considerable amount of water, is increased to a value in the range of about 10% to 20%.

For the purpose of complementing the purification, the xylose liquor evaporated previously is subjected to two other unit operations, aiming, essentially, to remove both organic and ionic contaminants still existing in the solution, which can harm seriously both the xylose hydrogenation and the crystallization cycles performed subsequently, regarding the xylose proper and the xylitol as well.

The treatment of the xylose solution requires a phase of clarification with active charcoal, in the approximate proportion of 10 g/100 ml, at a temperature comprised between 70° C. and 80° C. and during 60 minutes, without needing to adjust the pH of the medium, since already stabilized in the range of 6.0 to 7.0. Having been filtered, in order that the charcoal and the impurities adsorbed leave the main stream, this said stream passes through three successive beds of ion exchange resins—cationic, anionic and mixed resins—, undergoing an extensive deionization until its resistivity reaches a minimum value of 300,000 Ohm.cm.

At this point, the purified solution of xylose having a total dry solids concentration of between 10% and 20% by weight, and a xylose purity within the range of 65% and 85% by weight, on a dry solids basis, enters the third step of the route which is an object of the invention. By means of an evaporation under a vacuum in the range of 700 to 750 mm Hg, the dry solids content is adequately increased until it stabilizes at a value comprised between 75% and 85% by weight, this parametric region being considered ideal for transferring the solution to the crystallizers, in order to produce, with an appreciable yield, crystals of a regular habit, which are well-formed and homogeneous, and have a great purity, these characteristics being absolutely vital in order that the process be successful with respect to the commercialization of the xylose.

The crystallization of the xylose by the lowering of the temperature of its purified concentrated solution, in accordance with the invention, is carried out in four stages which include the controlled cooling of the medium and the addition of regulative nucleation seeds.

In the first stage, the solution in question, fed into the crystallizer with a concentration higher than 75% by weight and a xylose content preferably comprised between 65% and 85% by weight, on the dry basis, is promptly cooled from an initial temperature comprised between 55° C. and 65° C. to a temperature comprised between 45° C. and 52° C., depending on the degree of purity of the solution, according to a rate of heat transfer in the range of 1.0° C./h to 2.5° C./h.

Having accomplished the procedure described above, there is initiated the subsequent stage wherein there is carried out an isothermal seeding, i.e. at the same temperature as the temperature of the solution that has been reached at the end of the preceding stage, with xylose crystals exhibiting having a particle size distribution with a mean diameter in the range of 20 microns to 40 microns, for a period of time comprised between 30 minutes and 1 hour, and having a weight of pure xylose seed in relation to the weight of xylose in the solution comprised between 0.5% and 3.0% by weight, preferably comprised between 0.8% and 1.2% by weight.

The employment of the seed in this massive proportion, when compared with the mass of xylose in the medium, and having particles of small dimensions aims to provide a considerably large surface area for the growth of the crystals by the mechanism of surface integration, which consists in the bind of the free solute molecules to the faces of the crystals through a surface reaction, thereby permitting that the crystals become conveniently large for their applications and be more easily separated from the mother liquor during the centrifugation operation, at the end of the crystallization, thereby contributing to the attainment of very high yields of recovery of the xylose, which are characteristic of the process in accordance with the invention.

The third stage consists in the slow cooling of the massecuite according to a predetermined and automatically controlled temperature profile, through a critical zone of temperatures wherein the optimum supersaturation limit cannot be exceeded in order to prevent any spontaneous nucleation. With this purpose, therefore, the massecuite temperature is lowered progressively, at a moderate constant rate comprised between 0.2° C./h and 0.6° C./h, until it stabilizes at a level wherein the temperature is comprised between about 40° C. and about 42° C. During the course of this trajectory, the seed crystals grow appreciably, with the distinct formation of sharp edges, flat faces and well-delineated shapes.

The final stage of the process for the crystallization of xylose, herein identified as being the fourth one, consists in accomplishing a fast cooling of the massecuite produced in the preceding stage, according to a similarly predetermined and also automatically controlled temperature gradient, from its temperature reached at that point, within the range of 40° C. to 42° C., to a temperature of between about 25° C. and about 30° C., at a constant heat transfer rate of between 0.5° C./h and 1.5° C./h.

Throughout this stage of fast lowering of the temperature, wherein spontaneous nucleation is still avoided or at least reduced, the crystals grow substantially, removing from the liquid phase a large fraction of the solute therein remaining, until there is reached the target point in which the crystallization proper is completed after a supplementary period which serves to stabilize the temperature reached by the massecuite at the end of this final stage, comprised between 25° C. and 30° C. This value corresponds, at the same time, to the temperature at the termination of the crystallization process and to that of the centrifugation of the crystals, thereby resulting an overall crystallization cycle having a duration of between 36 hours and 60 hours, calculated by totaling each one of its constituent stages.

The recovery of the xylose crystals is made viable by means of centrifugation of the massecuite formed at the end of the crystallization profile, whereupon the crystallization medium is separated into a cake consisting of xylose crystals and a liquid phase with a residual xylose content, which is strategically recycled, with the impurities contained therein, so as to be incorporated into the wastes that are normally processed in the effluents treatment facility. The crystals are promptly washed with cold water in the centrifuge proper, to eliminate any fortuitous impurities adhered to their surface, thereby increasing even further the already high purity of the crystalline xylose.

At this point, in case there is interest in the direct commercialization of the xylose, the washed crystals, having a moisture content of between about 1% and about 5%, are carefully transferred to a rotary dryer, that employs dry air at a mean temperature of 100° C., aiming at obtaining a final product with moisture levels lower than or equal to 0.5%. Thus, the dried material is sized by screening, in order to have its granulometric characteristics adjusted according to the market specifications, and is then promptly packed in an environment with controlled temperature and humidity conditions. A quantity of xylose crystals approximately equal to, at most, 5% of the total mass is separated for the preparation of seed needed for new crystallization cycles, and is subjected to a grinding operation until such crystals attain dimensions preferably comprised between about 20 microns and about 40 microns.

The purity of the product obtained by the process according to the invention is excellent, being manifested by a content of xylose higher than 99.0% by weight and a content of glucose and arabinose, which are practically the only residual impurities, lower than 1.0% by weight, both on a dry solids basis. Furthermore, its microcrystalline particles exhibit a quite narrow size distribution, with a mean diameter within the range of about 150 microns and about 300 microns, which aspect reinforces the appropriate choice of the operating conditions employed in the crystallization of xylose.

However, in accordance with the central line that permeates this patent, in order that the route which was conceived be fully unfolded up to the desired formation of the xylitol, the xylose crystals resulting from the preceding centrifugation are dissolved in deionized process water, until there is established a total dry solids concentration in the range of 54% to 56% by weight. In this fourth step of the processing, such a solution is hydrogenated in the presence of Raney nickel catalyst, after the preliminary adjustment of the pH within 4.5 and 5.5, at a pressure of about 580 psig and at a temperature in the range of 145° C. to 155° C., the reaction being allowed to continue for an average time in the range of 80 minutes to 90 minutes, which is required for the conversion of approximately 98% to 99% of the xylose existing in the xylitol-containing medium.

Having been filtered, and being already completely free from the catalytic material, the raw hydrogenation solution enters the fifth step of the process, wherein it is initially fed into an adsorption column packed with granular active charcoal, at a temperature in the range of 65° C. to 75° C. and having a residence time properly determined, thereby occurring its clarification. Afterward, having been lowered its temperature to a value in the range of 43° C. to 47° C., the stream passes through a set of beds composed respectively of cationic, anionic and mixed resins, undergoing an extensive deionization until the resistivity at the battery outlet increases to a value in the range of 800,000 to 1,000,000 Ohm.cm, thereby becoming evident the high degree of purification aimed at the crystallization of the xylitol, since the presence of interfering substances in the said stream can harm the reproduction of the habit standards of the crystals.

Subsequently, the solution resulting from the ion exchange unit, with a dry solids content comprised between 52% and 54% by weight, and a xylitol purity within the range of 96% and 98% by weight, on a dry solids basis, passes through an evaporation system, operating under a vacuum of 700 mm Hg, wherein its concentration, by means of the loss of the excess mass of water, is increased to a value within the range of 70% to 75% by weight.

The last step of the route which is an object of the invention refers to the crystallization of the xylitol by the lowering of the temperature of its purified concentrated solution, and, as in the case of the xylose, is carried out in four phases which include the controlled cooling of the medium and the addition of crystallization seeds capable of attenuating the intensity of the phenomenon of spontaneous nucleation.

In the first phase, the said solution, fed into the crystallizer with a concentration higher than 70% by weight and a xylitol content preferably comprised between 96% and 98% by weight, on a dry solids basis, is immediately cooled from an initial temperature of between 58° C. and 60° C. to a temperature comprised between 48° C. and 52° C., according to the purity of the solution, by means of a rate of heat transfer in the range of 1.0° C./h to 2.0° C./h.

In the next phase, there is accomplished an isothermal seeding, i.e. without changing the temperature that has been reached at the end of the previous stage, with xylitol crystals having sizes comprised between 20 microns and 40 microns, for a period of time comprised between 30 minutes and 1 hour, and in the approximate proportion of between 0.5% and 3.0% by weight of pure xylitol seed crystals in relation to the mass of xylitol in the solution, preferably in the proportion of between 1.0% and 1.5% by weight.

As in the crystallization of the xylose, the employment of the seed in such a relatively large amount and with particles of reduced dimensions aims to provide a substantially large surface area for the perfect growth of the xylitol crystals by the aforementioned mechanism of surface integration, thereby allowing the said crystals to become adequately large for their applications and to be more easily separated from the mother liquor during the centrifugation operation, at the termination of the crystallization, thus contributing effectively to the attainment of very high yields of recovery of xylitol, which characterize the process which is detailed in this patent.

The third phase consists in the slow cooling of the massecuite in accordance with a predetermined and automatically controlled temperature gradient, through a critical zone of temperatures wherein the optimum supersaturation limit should not be exceeded so as to impede the occurrence of the spontaneous nucleation. Accordingly, the massecuite temperature is lowered progressively, at a moderate constant rate maintained between 0.7° C./h and 1.0° C./h, until it stabilizes at a level wherein its value is comprised between 35° C. and 40° C. During the course of this profile, the seed crystals grow pronouncedly, with a distinct formation of sharp edges, flat faces and well-delineated contours.

The final phase of the process for the crystallization of xylitol consists in accomplishing a fast cooling of the massecuite, according to a similarly predetermined and also automatically controlled temperature gradient, from its temperature at the end of the previous step, in the range of 35° C. to 40° C., to a temperature comprised between 19° C. and 23° C., at a constant rate of between 1.5° C./h and 2.0° C./h.

Throughout this phase of fast lowering of the temperature, having been still avoided or at least minimized the spontaneous nucleation, the crystals grow intensely, removing from the liquid phase a large proportion of the solute therein remaining, until the expected moment in which the crystallization proper reaches its termination, after having been elapsed a supplementary period of time which serves to stabilize the temperature at the end of this last phase, with a value comprised between 19° C. and 21° C. This value is not only the temperature at the end of the crystallization but also that one corresponding to the centrifugation of the crystals, thereby resulting an overall crystallization cycle with a duration of between 30 hours and 50 hours, calculated by totaling each one of its constituent phases.

The recovery of the crystalline xylitol is effected by means of centrifugation of the massecuite, whereby there are separated a cake of xylitol and a liquid phase which is rich in xylitol, which in turn is strategically recycled to be mixed with the purified solution of xylitol, prior to its evaporation, in order to improve substantially the overall yield of the process. The crystals are then promptly washed with cold water, in the centrifuge proper, to eliminate occasional contaminants adhered to their surface, thereby increasing even further the already remarkably high purity of the crystalline xylitol.

Immediately after, the washed crystals, having a moisture content in the range of about 2% to 3%, are then transferred to a rotary dryer that utilizes dry air at a mean temperature of about 90° C., with the purpose of ensuring that the product be obtained with moisture levels which are never higher than 0.1%. Thereafter, the dried material is sized by screening, so as to have its granulometric distribution adjusted in accordance with the market specifications, and is then packed in an environment with controlled temperature and humidity. A quantity of crystals equivalent to, at most, 5% of the entire mass is saved for the preparation of seed, which is needed in further crystallization cycles, and is subjected to a grinding operation until the respective crystals attain dimensions comprised between about 20 microns and about 40 microns.

The purity of the xylitol manufactured in accordance with the present invention is excellent, being demonstrated by a minimum xylitol content equal to 99.5% by weight and a content of arabitol, which is practically the only residual impurity detected, lower than 0.5% by weight, both on the dry basis, in the final product to be sold to their consumers. Moreover, their microcrystalline particles exhibit a very narrow size distribution, with a mean diameter within the range of about 400 microns and about 600 microns, this being an aspect which corroborates the precise selection of the conditions employed in the crystallization of xylitol.

Even though the prior art presents a large number of technical difficulties intrinsic to the process for crystallization of the xylose, and, by the same token, of the xylitol, the present route revealed itself, surprisingly, as a profitable source of important technical novelties and decisive improvements introduced by the Applicant.

The conjunction of the technical improvements incorporated into the processing of the xylose and the xylitol, based on the innovative content of the invention, brought about, as essential effects, a very high purity of the final products, as well as excellent morphological and granulometric characteristics of the respective crystals, which were achieved by means of the elimination or attenuation of the undesirable phenomenon of spontaneous nucleation, with associated advantages related to a considerably short length of time for the crystallization cycles, a high yield of recovery of such products and a superbly improved overall economics of the process.

Reiterating these positive aspects, the reading of the comments incorporated into the paragraphs which follow permits the adequate comprehension of the efforts made for the optimization of the physical and functional properties of the crystalline xylose and the crystalline xylitol, obtained by the process according to the present invention.

The granulometric analysis performed on the particulate material which constitutes the crystalline xylose and the crystalline xylitol reveals that such products possess a narrow and centered particle size distribution, with a mean diameter comprised, in the first case, between about 150 microns and about 300 microns, having approximately 90% of its particles within this size range, while, in the second case, 85% of the crystals are concentrated in the range of 400 microns to 600 microns.

As regards the xylose, such characterization is complemented through the results which express the relative percentage by weight of particles of each of the different size fractions represented in a given sample, as follows: 0.5% over 840 microns; 4% over 420 microns; 27% over 250 microns; 53% over 177 microns; and 15.5% between 177 microns and 125 microns.

As for the xylitol, by expressing similarly its granulometric characterization in percentage terms, by weight, in a typical sample space, it is possible to note the narrow limits of variation of the dimensions of the crystals which are formed by means of the route structured in this patent: 2% over 840 microns; 62% over 420 microns; 28% over 250 microns; 7% over 177 microns; and 1% between 177 microns and 125 microns.

Another pronounced physical characteristic of both the crystalline xylose and the crystalline xylitol, produced in accordance with the invention, for a particle size cut within the range of 100 microns and 800 microns, is their apparent density, comprised, in the first case, between about 0.52 g/l and about 0.58 g/l, preferably comprised between about 0.54 g/l and about 0.56 g/l, and, in regard to the xylitol, comprised between about 0.48 g/l and about 0.54 g/l, preferably comprised between about 0.50 g/l and about 0.52 g/l.

As regards the applications for the products derived from the inventiveness achieved by the Applicant, with respect to both the crystalline xylose and the crystalline xylitol, the hygroscopicity and the dissolution time reflect, as functional specifications, the pronounced performance levels attained in consequence of the processing route herein minutely detailed.

The first of these functional specifications relates to the tendency that the crystalline particles have to absorb moisture from the air, a relatively high hygroscopicity being a factor capable of imposing a serious restriction on the desired fluidity of the material, insofar as the moisture absorbed agglutinates the pulverulent solid and thereby practically impedes the free flow of its particles. Hygroscopicity is defined as the proportion by weight represented by the water absorbed by a sample of the particulate product which is kept in a hygrostat at a constant relative humidity of 80%, for 24 hours.

When subjected to this test, the crystalline xylose according to the invention exhibits a hygroscopicity which is less than about 2%, preferably not greater than 1.8% and, still more preferably, less than about 1.5%, while the crystalline xylitol derived therefrom has a hygroscopicity which is less than about 1.8%, preferably less than about 1.6% and, still more preferably, less than about 1.3%.

These values demonstrate that the two said products, in accordance with the technology developed by the Applicant, are outstanding ones owing to the considerably low relative levels of hygroscopicity, thereby revealing their high physicochemical stability which results from the extremely high purity and the well-defined morphology conferred on their crystals by the processing, thereby allowing, advantageously, their better handling, packing, storage, transportation and final utilization, insofar as there is maintained an excellent fluidity, which delays the chances of caking the material, for longer periods of time.

The other functional particularity associated with the crystalline xylose and the crystalline xylitol, according to the present invention, is represented by the dissolution time, expressed in seconds, which denotes the facility that a given amount of the particulate material has for dissolving completely in a predetermined amount of water, under specific conditions of agitation and temperature, thereby forming a perfectly clear or transparent solution. This parameter is estimated by means of a specific test which consists in introducing 5 grams of a granulometric cut, within the range of 100, microns to 5.95 microns, of the product to be tested into 150 g of demineralized and degassed water maintained at 20° C. and subjected to stirring at 200 rpm in a 250 ml low form beaker, the dissolution time being the time necessary, after introduction of the granulometric cut, to obtain perfect visual clarity of the suspension thus prepared.

Within the bounds of the processing route conceived in this patent, the crystalline xylose exhibits a dissolution time which is less than 18 seconds, more preferably less than 15 seconds, while the crystalline xylitol, subjected to an analogous test, does not exceed characteristically the 20 second mark, remaining preferably under 18 seconds. These values in question reveal that the products according to the invention dissolve very quickly in water, not only owing to their high solubility levels, but also to their peculiar size distributions of the crystals, which contribute strongly to the dissolution of their microcrystalline granules, making them extremely appropriate for countless industrial applications.

In summary, the invention presented by the Applicant, in virtue of the innovations introduced into the several different steps of its structure, constitutes, undeniably, a singular alternative of manufacturing xylose and xylitol, since it enables a combination of strategic, technical, economic, and commercial factors which is distinctly different from and much better than those utilized by the prior art. Thus, such technical novelties and improvements incorporated into the process in accordance with the invention bring about very important technical effects, among which effects stand out the reduction in the level of technical difficulty and in the costs of the purification of the xylose solution extracted from the sugar cane bagasse; the suppression or attenuation of spontaneous nucleation in the crystallization profiles; the high yields of recovery of the final products; the highly improved economics of the process in all of its six constituent steps; and the production of xylose and xylitol crystals of high purity, and with morphological, granulometric and functional characteristics absolutely adequate for the market needs.

With the specific purpose of facilitating a full understanding of the invention herein explained, it is worth illustrating it by means of the following example, which is typical of the preparation of the crystalline xylose and the crystalline xylitol, and which in no way restricts or harms the breadth of the scope of the original technology systematized in accordance with the work developed by the Applicant.

### EXAMPLE

Preparation of Crystalline Xylose and Crystalline Xylitol Conducted in Accordance With the Processing Route of the Present Invention.

A feed of sugar cane bagasse is ground until 95% of its particles attain a size of less than 3 millimeters and, subsequently, is subjected to an exhaustive washing using water at 90° C., in the proportion of 8 parts of water per part of bagasse.

After the separation of the liquid phase, the bagasse is then fed into a hydrolysis reactor, containing a previous amount of water ballast and undergoing an ensuing addition of a 98% sulfuric acid solution, so as to adjust the pH of the medium to 1.5, thereby forming a suspension with a dry solids content of 15% by weight. The reaction proceeds at a temperature of 140° C., for 120 minutes, until there is obtained a solution of xylose having a dry solids concentration of 2.5% and a xylose purity equivalent to 65%, on a dry solids basis.

At the end of the hydrolytic process, the solution is neutralized with an 8% suspension of calcium hydroxide to a pH of 6.5, and is then dosed with ferric chloride and an anionic polyelectrolyte. Only then there is accomplished the dewatering of the bagasse, with the separation of the moist solid residue, thereby resulting a partially purified liquor, with a dry solids content equal to 2.5% and a purity of 65%, on a dry solids basis, which is immediately fed into an evaporation unit, operating under a vacuum of 720 mm Hg, so as to be removed a significant fraction of water, until its concentration reaches a value of 15%.

The complementary treatment of the said xylose solution, in the course of the example herein minutely detailed, takes place by employing active charcoal in the proportion of 10 g/100 ml, at a temperature of 75° C. and for, at least, 60 minutes, after which the stream passes through a battery of ion exchange beds—cationic, anionic and mixed-resin—until its resistivity reaches the satisfactory value of 400,000 Ohm.cm.

Having already been freed from undesirable organic and ionic contaminants, the purified solution undergoes another evaporation, in a system operated under a vacuum of 720 mm Hg, until a dry solids content of 82% by weight is attained, thereby maintaining a xylose purity equivalent to 80% by weight, on a dry solids basis.

The step for the crystallization of the xylose is initiated by cooling the concentrated solution from a temperature of 60° C. to 50° C., according to a constant rate of heat transfer equal to 2.0° C./h. At the final temperature of this stage, there is accomplished at once the seeding of the medium with xylose crystals having particles sizes in the range of 20 microns to 40 micron, in the proportion of 1.0% by weight in relation to the mass of xylose in the solution, over a period of 60 minutes.

The preparation proceeds with a further and slow decrease in the temperature until the limit of 42° C., under a gradient equivalent to 0.5° C./h, and reaches its final stage with the adoption of a heat transfer rate equal to 0.8° C./h, until the temperature is treated to the lower level of 28° C., corresponding to the value registered in the centrifugation of the massecuite, thereby amounting to an overall crystallization cycle of 40 hours. After having been washed, the xylose crystals come into contact with dry air at a temperature of 100° C., whereby the moisture content of the product is reduced from 3% to 0.3%, and are than passed through a particle-size classifier so as to fulfill the market requirements, and furthermore attending to the separation of a certain quantity of the material for the preparation of the crystallization seeds.

The crystalline xylose of high purity, manufactured in accordance with the conditions employed in the example, exhibited the following characteristics:
a xylose content of 99.7% by weight, on a dry solids basis;
a content of glucose plus arabinose of 0.3% by weight, on a dry solids basis;
a residual moisture content of 0.3% by weight;
an apparent density of 0.55 g/l;
the following typical granulometric spectrum:
   particles with a size greater than 840 microns: approximately 0.5%;
   particles with a size greater than 420 microns: approximately 4.0%,
   particles with a size greater than 250 microns: approximately 27.0%;
   particles with a size comprised between 177 microns approximately 53.0%;
   particles with a size comprised between 177 microns and 125 microns: approximately 15.5%;
   a mean diameter approximately equal to 200 microns;
a hygroscopicity of 1.5%, at relative humidity of 80%; and
   a solution time of 15 seconds.

The portion of the said material which has not been utilized directly in the form of crystalline xylose continues in the processing route developed by the Applicant, thereby entering the subsequent step through the dissolution of the pulverulent material in deionized water in order that the dry solids concentration attains a value of 55%. This recently made solution is hydrogenated using a Raney nickel catalyst, in a medium having a pH adjusted to a value of 5.0, at a temperature of 150° C. and at a pressure of 580 psig, for a sufficiently long period of time of about 90 minutes, so that the tests for the control of product quality verify that the level of conversion of xylose to xylitol has reached the value of 98%.

The raw hydrogenation solution, after being filtered and without any particles of the catalyst, flows through a column packed with granular active charcoal, at a temperature of 70° C., and is cooled shortly afterward to 45° C. aiming at its deionization in a set of cationic a and mixed-resin beds, until a resistivity value of 1,000,000 Ohm.cm is reached, thereby confirming the elimination of fortuitous interfering constituents for the subsequent steps of the process.

The solution obtained in the ion exchange unit, with a dry solids content of 53% by weight and a xylitol purity equivalent to 97% by weight, on a dry solids basis, is subjected to another evaporation, in a unit operated under a vacuum of 100 mm Hg, thereby increasing the concentration to 73% by weight, being maintaiind the xylitol purity at the value equivalent to 97% by weight, on a dry solids basis.

The crystallization profile of the xylitol is initiated with the, lowering of the temperature of the concentrated solution from 60° C. to 50° C., according to a constant rats of heat transfer equal to 10° C./h. At the end of this stage, there is promptly accomplished the seeding of the medium with xylitol seed crystals having particle sizes within the range of 20 microns to 40 microns, in the proportion of 1.5% by weight in relation to the mass of xylitol in the solution, for a period of 1 hour.

The unit operation proceeds with another slow decrease in the temperature until there is attained the value of 38° C., subject to a thermal gradient of 0.7° C./h, and reaches its final stage by employing a heat transfer rate of 1.5° C./h, until there is established a lower limit of temperature of 20° C., corresponding to that which was registered in the centrifugation of the massecuite; thereby amounting to an overall crystallization cycle of 40 hours.

After the washing, which is carried out still in the centrifuges, the xylitol crystals are brought into contact with dry air at a temperature of 90° C., thereby reducing the moisture content of the product from 2% to 0.08%, and pass through a screening unit, aiming at the adjustment of their granulometric distribution in accordance with the requirement of the industrial applications of interest, without neglecting to reserve a certain amount of the material for preparing the seeds.

The crystalline xylitol of high purity, manufactured by the process according to the invention, and in accordance with the conditions employed in the present example, exhibited the following characteristics:
a xylitol content of 99.9% by weight, an a dry solids basis;
an arabitol content of 0.1% by weight, on a dry solids basis;
a residual moisture content of 0.08% by weight;
an apparent density of 0.51 g/L;
the following typical granulometric-spectrum:
   particles with a size greater than 840 microns:
      approximately 2.0%;
   particles with a size greater than 420 microns;
      approximately 62.0%;
   particles with a size greater than 250 microns:
      approximately 26.0%;
   particles with a size greater than 177 microns:
      approximately 7.0%;,
   particles with a size comprised between 177 microns and 125 microns: approximately 1.0%;
a mean diameter approximately equal to 450 microns;
is a hygroscopicity of 1.3%, at a relative humidity of 80%; and
a dissolution time of 18 seconds.

These numbers indicate that the crystalline xylose and the crystalline xylitol, produced according, to the route adopted in the example under discussion, possess physical and functional characteristics which are very well adjusted to the needs of the market. Furthermore, the yields of recovery of the xylose and the xylitol, for the process parameters and operating conditions employed herein, attained the various of 65% and 98%, respectively, thereby corroborating, unquestionably, the high efficiency of the process derived from the inventiveness achieved by the Applicant.

1. Process for the production of crystalline xylose characterized by the fact that the raw material is sugar cane bagasse and that its route comprises the following steps:
(a) in a first step, the sugar cane bagasse is ground, in order that only 5% of its particles have dimensions greator than 3 millimeters, and is subjected to an exhaustive washing with water at a minimum temperature of 80° C., in the proportion of between 5 and 10 parts of water per part of bagassa; by weight. Subsequently, upon completion of the separation of the liquid phases the bagasse is then fed into a hydrolysis reactor, having a water ballast dosed with a solution of sulfuric acid of 98% in concentration, so as to adjust the pH of the medium in the range of 1.0 to 2.0, thereby forming a suspension with a dry solids content within the range of 10% to 20% by weight. The reaction proceeds at a temperature of between 120° C. and 150° C., for approximately 120 minutes, until there is obtained a xylose solution having a dry solids concentration within the range of 2% to 6% by weight and a xyloss purity in the range of 60% to 75%, on a dry solids basis; and
(b) in a second step, the liquor formed at the end of the hydrolysis is neutralized with a suspension of calcium hydroxide having a concentration of between 5% and 10%, thus raising the pH of the medium to the region comprised between 6.0 and 7.0, and is then treated with ferric chloride and an anionic polyelectrolyte. After the separation of the solid residue, the partially purified liquid is concentrated in an evaporation unit operating under a vacuum of between 700 mm Hg and 750 mm Hg, so that the dry solids content reaches the range of 10% to 20% by weight, the xylose purity being maintained between 60% and 75% by weight, on a dry solids basis, whereupon the treatment of the solution proceeds, first by means of a clarification with active charcoal, in the approximate proportion of 1.0 g/100 ml, at a temperature of between 70° C. and 80° C. and during 60 minutes, and then through a contiguous operation of deionization conducted in beds containing cationic, anionic and mixed resins, until its resistivity attains a value of equal to or greater than 300,000 Ohm.cm; and
(c) in a third step, the completely purified solution is concentrated again, in an evaporation unit operating under a vacuum of between 700 mm Hg and 750 mm Hg, so that its dry solids content be promptly raised to a value in the range of 75% to 85% by weight, with a xylose purity of between 65% and 85% by weight, on a dry solids basis. Thereafter, the crystallization profile of the xylose proceeds, by means of the lowering of the tmmpmrature of the medium, according to four distinct stages, preliminarily by decreasing the temperature from the range of 55° C. to 65° C. to the range of 45° C. to 52° C., in accordance with a rate of heat transfer in the range of 1.0° C./h. to 2.5° C./h. Hereupon, at the temperature at the end of the preceding stage, there is accomplished the necessary seeding of the medium with crystals of xylose, having a granulometric distribution of between 20 microns and 40 microns, over a period of between 30 minutes and 1 hour, and in the approximate proportion of between 0.5% and 3.0% by weight of pure xylose seod crystals in relation to the mass of xylose in the solution, preferably in the proportion of between 0.8 and 1.2% by weight. Subsequently, a new cooling stage brings, about a very slow decrease in the temperature from the range of 45° C. to 52° C. to the range of 40° C. to 42° C., by means of a thermal gradient in the range 0.2° C./h to 0.6° C./h, whereupon there, follows a final phase of fast lowering of the temperature, with a heat transfer rate comprised between 0.5° C./h and 1.5° C./h, until the stabilization of its value in a lower limit comprised between 25° C. and 30° C. Upon completion of the crystallization, with an overall cycle having a duration in the range of about 3.6 hours to 60 hours, the processing efforts are then directed towards the centrifugation of the massecuits, the washing of the xylose crystals, their ensuring drying through direct contact with dry air at a temperature of 100° C., thereby decreasing the moisture content from within the range of 1% and 5% to 0.5%, the screening of the material to adjust its size distribution so as to meet the requirements of the market and its packing under controlled conditions and in adequate packaging, without neglecting to separate a certain fraction of the crystallization product for the preparation of the crystallization seeds.

2. Process according to claim 1, characterized by the fact that the solution of xylose obtained at the end of the last purification operation has a xylose content comprised between 65% and 85% by weight, on a dry solids basis, and more preferentially comprised between 75% and 85% by weight, on a dry solids basis.

3. Process according to claim 1, chararacterizad by the fact that the solution of xylose obtained at the end of the last purification operation has a dry solids concentration preferably comprised between 75% and 85% by weight.

4. Process according to claim 1, characterized by the fact that the solution of xylose used as the feed for the crystallization has a xylose content preferably comprised between 75% and 85% by weight, on a dry solids basis.

5. Process acording to claim 1, characterized by the fact that the solution of xylose used as the feed for the crystallization has a dry solids concentration preferably comprised between 75% and 85% by weight.

6. Process according to claim 1, characterized by the fact that the proportion of the mass of added xylose seed in relation to the mass of xylose in the solution is comprised between about 0.5% and about 3.0% by weight.

7. Process according to claim 1, characterized by the fact that the xylose seed crystals have sizes preferably comprised between about 20 microns and about 40 microns.

8. Process according to claim 1, characterized by the fact that the seed is prepared from about 2% to about 5% of the mass of crystals of the crystalline xylose produced by the process proper, in a previous crystallization cycle.

9. Crystalline xylose of high purity produced by the process according to any one of claims 1 to 8, characterized by the fact that it has crystalline microgranules composed of crystals possessing a xylose content higher than or equal to 99.0% by weight, on a dry solids basis; a content of glucose plus arabinose lower than 1.0% by weight, on a dry solids basis; a residual moisture content lower than 0.5% by weight; an apparent density comprised between about 0.52 g/l and about 0.58 g/l, for a granulometric cut within the range of 100 microns to 800 microns; approximately 0.5% of the particles with a size greater than 840 microns; approximately 4% of the particles with a size greater than 420 microns; approximately 27% of the particles with a size greater than 250 microns; approximately 53% of the particles with a size greater than 177 microns; approximately 15.5% of the particles with a size comprised between about 177 microns and about 125 microns; a mean particle diameter comprised between about 150 microns and about 300 microns; a hygroscopicity lower than 2.0%, when determined at a relative humidity of 80%; and a dissolution time less than approximately 18 seconds.

10. Crystalline xylose of high purity according to claim 9, characterized by the fact that it has a xylose content preferably higher than 99.0% by weight, on a dry solids basis, and more preferentially higher than 99.2% by weight, on a dry solids basis.

11. Crystalline xylose of high purity according to claim 9, characterized by the fact that it has a content of glucose plus arabinose preferably lower than 1.0% by weight, on a dry solids basis, and more preferentially lower than 0.8% by weight, on a dry solids basis.

12. Crystalline xylose of high purity according to claim 9, characterized by the fact that it has a residual moisture content preferably lower than about 0.5.% by weight and more preferentially lower than about 0.3% by weight.

13. Crystalline xylose of high purity according to claim 9, characterized by the fact that it has an apparent density preferably comprised between about 0.52 g/l and about 0.58 g/l and more preferentially comprised between about 0.54 g/l and about 0.56 g/l, for a granulometric cut within the range of about 100 microns to about 800 microns.

14. Crystalline xylose of high purity according to claim 9, characterized by the fact that it has a hygroscopicity, determined at a relative humidity of 80%, preferably lower than about 2.0% and more preferentially lower than about 1.8%.

15. Crystalline xylose of high purity according to claim 9, characterized by the fact that it has a dissolution time preferably lower than approximately 15 seconds.

16. Process for the production of crystalline xylitol characterized by the fact that the raw material is the crystalline xylose obtained from sugar cane bagasse, according to the previous claim 1, and that its route comprises the following steps:
(a) in a first step, the crystalline xylose is dissolved in deionized water, thereby forming a solution with a dry solids concentration in the range of 54% to 56% by weight, and subsequently, insofar as the pH of the medium has been adjusted to be in the range of 4.5 to 5.5, is hydrogenated in the presence of a Raney nickel catalyst, at a pressure of about 580 psig and at a temperature of between 145° C. and 155° C., for an average time in the range of 80 minutes to 90 minutes, until 98% to 99% of the xylose is converted to xylitol; and
(b) in a second step, the filtered raw hydrogenation solution, free from the catalyst particles and at a temperature of between about 65° C. and about 75° C., passes through a column containing granular active charcoal, whereupon it undergoes, after being accomplished a reduction in the stream temperature to a value of between 43° C. and 47° C., a deionization operation in a set of cationic, anionic and mixed-resin beds, until its resistivity attains a value comprised between 800,000 and 1,000,000 Ohm.cm. Thereafter, in an evaporation system operating under a vacuum of 700 mm Hg, the concentration of the solution being of between 52% and 54% by weight is increased to a value comprised between the limits of 70% and 75% by weight, the xylitol purity being maintained between 96% and 98%; and
(c) in a third and last step, the deionized concentrated solution of xylitol, having a dry solids content of between 70% and 75% by weight and a xylitol purity of between 96% and 98% by weight, on a dry solids basis, initiates the crystallization profile by lowering the temperature of the medium, going through four different stages, preliminarily with the decrease in the temperature from the range of 58° C. to 60° C. to the region of 48° C. to 52° C., under a heat transfer rate of from about 1.0° C./h to 2.0° C./h. At this point, at the temperature at the end of the previous stage, there is achieved the appropriate seeding of the medium with crystals of xylitol, having a granulometric distribution of between 20 microns and 40 microns, for a period of between 30 minutes and 1 hour, and in the approximate proportion of between 0.5% and 3.0% by weight of pure xylitol seed crystals in relation to the mass of xylitol in the solution, preferably in the proportion of from about 1.0% to 1.5% by weight. Afterward, an additional cooling stage brings about a slow decrease in the temperature from the range of 48° C. to 52° C. to the range of 35° C. to 40° C., according to a thermal gradient of from about 0.7° C./h to 1.0° C./h, whereupon there follows a final phase of fast lowering of the temperature, under a heat transfer rate comprised between 1.5° C./h and 2.0° C./h, until the stabilization of its value in a lower limit comprised between 19° C. and 23° C. On completion of the crystallization step, with an overall cycle having a duration of from about 30 hours to 50 hours, the preparations are then directed towards the centrifugation of the massecuite, the careful washing of the xylitol crystals with water, their subsequent drying through direct contact with dry air at a temperature of 90° C., thereby reducing the moisture content of the material from the range of 2% to 3% to the value of 0.1%, more preferentially lower than 0.1%, the screening of the material so as to conform its size distribution to the requirements of the consumers and its packing under controlled conditions and in adequate packaging, without neglecting to separate a certain fraction of the crystalline product for the preparation of the crystallization seeds.

17. Process according to claim 16, characterized by the fact that the solution of xylitol utilized as the feed for the crystallization has a xylitol content preferably comprised between 96% and 98% by weight, on a dry solids basis.

18. Process according to claim 16, characterized by the fact that the solution of xylitol utilized as the feed for the crystallization has a dry solids concentration preferably comprised between 70% and 75% by weight.

19. Process according to claim 16, characterized by the fact that the proportion of the mass of added xylitol seed in relation to the mass of xylitol in the solution is comprised between about 0.5% and about 3.0% by weight.

20. Process according to claim 16, characterized by the fact that the xylitol seed crystals have sizes preferably comprised between about 20 microns and about 40 microns.

21. Process according to claim 16, characterized by the fact that the seed is prepared from about, at most, 5% of the mass of crystals of the crystalline xylitol produced by the process proper, in a preceding crystallization cycle.

22. Crystalline xylitol of high purity produced by the process according to any one of claims 16 to 21, characterized by the fact that it has crystalline microgranules composed of crystals with a xylitol content higher than or equal to 99.5% by weight, on a dry solids basis; an arabitol content lower than 0.5% by weight, on a dry solids basis; a residual moisture content lower than 0.1% by weight; an apparent density comprised between about 0.48 g/l and about 0.54 g/l, for a granulometric cut within the range of 10.0 microns to 800 microns; approximately 2% of the particles with a size greater than 840 microns; approximately 62% of the particles with a size greater than 420 microns; approximately 28% of the particles with a size greater than 25.0 microns; approximately 7% of the particles with a size greater than 177 microns; approximately 1% of the particles with a size comprised between about 177 microns and about 125 microns; a mean particle diameter comprised between about 400 microns and about 600 microns; a hygroscopicity lower than 1.8%, when determined at a relative humidity of 80%; and a dissolution time less than approximately 20 seconds.

23. Crystalline xylitol of high purity according to claim 22, characterized by the fact that it has a xylitol content preferably higher than 99.5% by weight, on a dry solids basis, and more preferentially higher than 99.8% by weight, on a dry solids basis.

24. Crystalline xylitol of high purity according to claim 22, characterized by the fact that it has an arabitol content preferably lower than 0.5% by weight, on a dry solids basis, and more preferentially lower than 0.2% by weight, on a dry solids basis.

25. Crystalline xylitol of high purity according to claim 22, characterized by the fact that it has a residual moisture content preferably lower than about 0.1% by weight and more preferentially lower than about 0.08% by weight.

26. Crystalline xylitol of high purity according to claim 22, characterized by the fact that it has an apparent density preferably comprised between about 0.48 g/l and about 0.54 g/l and more preferentially comprised between about 0.50 g/l and about 0.52 g/l, for a granulometric cut within the range of about 100 microns to about 800 microns.

27. Crystalline xylitol of high purity according to claim 22, characterized by the fact that it exhibits a hygroscopicity, determined at a relative humidity of 80%, preferably lower than about 1.6% and more preferentially lower than about 1.3%.

28. Crystalline xylitol of high purity according to claim 22, characterized by the fact that it has a dissolution time preferably lower than approximately 18 seconds.
